(19) United States
(12) Patent Application Publication
(10) Pub. No.: US 2021/0054326 A1
(43) Pub. Date: Feb. 25, 2021

(54) REACTOR FOR CELL GROWTH

(71) Applicant: Alliance for Sustainable Energy, LLC, Golden, CO (US)

(72) Inventor: Nicholas Anthony SWEENEY, Lakewood, CO (US)

(21) Appl. No.: 16/998,045

(22) Filed: Aug. 20, 2020

Related U.S. Application Data
(60) Provisional application No. 62/890,097, filed on Aug. 22, 2019.

(51) Int. Cl. C12M 1/04; C12M 1/00

(52) U.S. Cl. CPC C12M 27/04; C12M 31/02; C12M 23/38

(57) ABSTRACT

The present disclosure relates to a reactor that includes a cylinder integrally connected to a top portion and a bottom portion, where the bottom portion is rounded and configured to contain a stir bar, the top portion includes a neck configured to receive a cap, the cylinder has a height (H) and a width (W) such that the ratio of H to W is between about 1:1 and about 10:1, and the cylinder is transparent to light having a wavelength between about 400 nm and about 700 nm.

### CROSS-REFERENCE TO RELATED APPLICATIONS

This application claims priority from U.S. Provisional Patent Application No. 62/890,097 filed on Aug. 22, 2019, the contents of which are incorporated herein by reference in its entirety.

### CONTRACTUAL ORIGIN

This invention was made with government support under Contract No. DE-AC36-08GO28308 awarded by the Department of Energy. The government has certain rights in the invention.

### SUMMARY

An aspect of the present disclosure is a reactor that includes a cylinder integrally connected to a top portion and a bottom portion, where the bottom portion is rounded and configured to contain a stir bar, the top portion includes a neck configured to receive a cap, the cylinder has a height (H) and a width (W) such that the ratio of H to W is between about 1:1 and about 10:1, and the cylinder is transparent to light having a wavelength between about 400 nm and about 700 nm.

In some embodiments of the present disclosure, the ratio may be between about 2:1 and about 6:1. In some embodiments of the present disclosure, H may be between about 5.5 inches and about 14.5 inches. In some embodiments of the present disclosure, W may be between about 2.0 inches and about 5.5 inches. In some embodiments of the present disclosure, the neck may have a height between 1.0 inch and 2.0 inches. In some embodiments of the present disclosure, the neck may have a width of about 2 inches. In some embodiments of the present disclosure, the cylinder may be constructed of at least one of glass, plexiglass, quartz, and/or a plastic. In some embodiments of the present disclosure, the reactor is capable of withstanding a temperature between about 100° C. and about 130° C. In some embodiments of the present disclosure, the cylinder may have a wall thickness of about 0.14 inches.

An aspect of the present disclosure is a reactor system that includes a reactor that includes a cylinder integrally connected to a top portion and a bottom portion, where the bottom portion is rounded, the top portion comprises a neck, the cylinder has a height (H) and a width (W) such that the ratio of H to W is between about 1:1 and 10:1, and the cylinder is transparent to light having a wavelength between about 400 nm and about 700 nm. The system also includes a stir bar positioned in the bottom portion, a cap positioned within the neck, and a first tube positioned in a first port passing through the cap into the reactor.

In some embodiments of the present disclosure, the system may further include a light source configured to provide light having a wavelength between about 400 nm and about 700 nm. In some embodiments of the present disclosure, a distal end of the first tube may be positioned in the bottom portion. In some embodiments of the present disclosure, the system may further include a second tube positioned in a second port passing through the cap into the reactor. In some embodiments of the present disclosure, a distal end of the second tube may be positioned in the bottom portion. In some embodiments of the present disclosure, the system may further include a third tube positioned in a third port passing through the cap into the reactor. In some embodiments of the present disclosure, a distal end of the third tube may be positioned in the top portion. In some embodiments of the present disclosure, the stir bar may be magnetic. In some embodiments of the present disclosure, the system may further include a stirring mechanism that is magnetically coupled to the stir bar and drives rotation of the stir bar.

An aspect of the present disclosure is a method that includes positioning a mixture that includes an algae and a growth medium in a reactor, agitating the mixture using a mixer positioned in the reactor at a speed between greater than zero RPM and about 1000 RPM, providing a gas to the mixture, and illuminated the mixture using a light source positioned externally to the reactor. In some embodiments of the present disclosure, the light source may be configured to provide at least one of a steady light or a fluctuating light.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 illustrates a reactor, according to some embodiments of the present disclosure.

FIG. 2 illustrates a reactor system that utilizes a reactor as described herein, according to some embodiments of the present disclosure.

FIG. 3 illustrates a reactor system utilizing six reactors as described herein, according to some embodiments of the present disclosure.

FIGS. 4A and 4B illustrate various exemplary geometries and dimensions of reactors, according to some embodiments of the present disclosure (all dimensions in inches).

### REFERENCE NUMBERS

100 . . . reactor unit
110 . . . reactor
112 . . . cylinder
113 . . . top portion
114 . . . bottom portion
115 . . . neck
116 . . . height
117 . . . width
118 . . . internal volume
120 . . . cap
125 . . . port
130 . . . tube
140 . . . mixer
200 . . . reactor system
210 . . . reaction medium
220 . . . light source
230 . . . light
300 . . . reactor array

### DETAILED DESCRIPTION

The embodiments described herein should not necessarily be construed as limited to addressing any of the particular problems or deficiencies discussed herein.

References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The present disclosure relates to reactors and reactor systems for growing biomass, for example algae, yeast, bacterial cells, and/or mammalian cells. Biomass settling negatively impacts growth and productivity. Some embodiments of the present disclosure combine, among other things, a reactor design with a mixer to address this issue, resulting in higher biomass productivity, better reproducibility, and improved ease of use. In some embodiments of the present disclosure, a mixer may be a magnetic stir bar that is positioned in a rounded bottom portion of a reactor, which enables the mixer to self-correct when the mixer moves off-center (e.g. relative to the long axis of the reactor). In some embodiments of the present disclosure, a reactor unit may be constructed to have the following features: A reactor constructed of a clear glass cylinder having a volume of about 2.6 liters and a round bottom portion and a top portion with a neck having at least on port, which may be used as an inlet port, and outlet port, and/or a sample port. In some embodiments of the present disclosure, the neck may be plugged with a cap (e.g. a rubber stopper) having one or more ports with a tube passing through each port, for example, one for a gas input into the reactor, one for a gas exiting the reactor, and/or one configured as a sample port enabling easy sampling.

In some embodiments of the present disclosure, an oblong-shaped magnetic stir bar may be positioned in a reactor, such that the stir bar (i.e. mixer) articulates perfectly within the rounded bottom portion of the reactor. In some embodiments of the present disclosure, a magnetic stir bar may have a length between 0.5 inches and three inches. When placed on a stir plate (magnetically coupled to the stir bar) the stir bar may self-correct to the center of the reactor and achieve up to 1000 RPM. This approach to biomass cultivation is easier to set up and, in some cases, provides better biomass productivity due to the unique geometry of the reactor. The reactor design is easier to use since the stir bar self-corrects leading to fewer compromised experiments, which happens often using incumbent reactor and/or mixing technology. In some embodiments of the present disclosure, for example when scaling up to larger sized reactors, mixers other than stir bars may be utilized, for example rotating shaft impeller mixers of various types such as paddle blades, ribbon blades, flat blade turbine mixers, anchor blades, spiral propeller blades, etc.

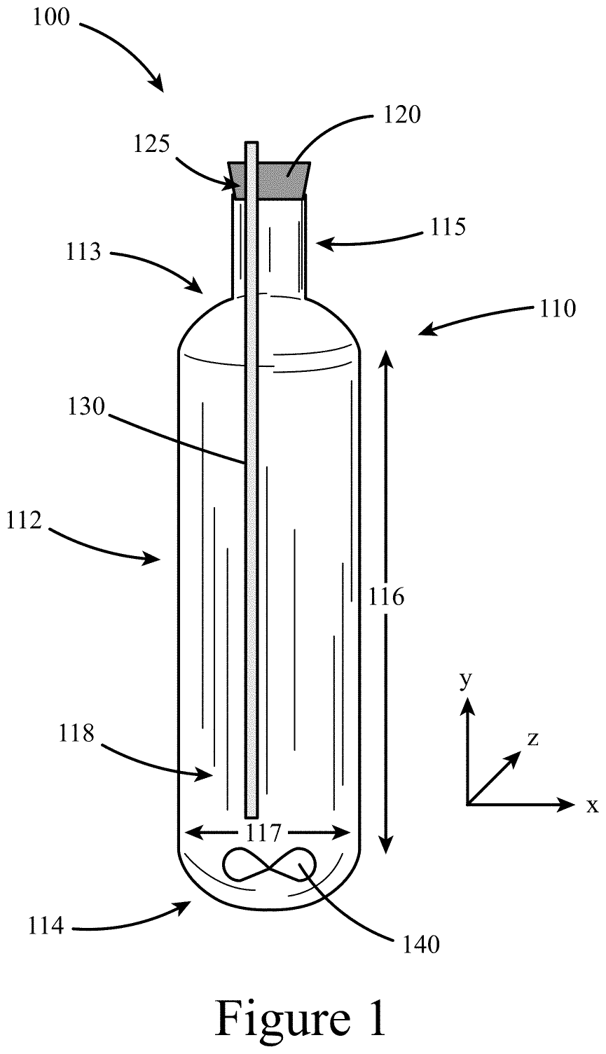

FIG. 1 illustrates a reactor unit 100, according to some embodiments of the present disclosure. The reactor unit 100 includes a reactor 110 constructed of three parts integrally connected: a cylinder 112 positioned between a top portion 113 and a bottom portion 114. The cylinder 112 has a height 116 and a width 117, positioned along the y-axis and within the xz-plane, respectively. The cylinder 112 may have a substantially circular and/or elliptical cross-section (in the xz-plane). However, in some embodiments of the present disclosure a reactor 110 may have a non-circular cross-section in the xz-plane; e.g. square, rectangular, triangular, hexagonal, and/or any other suitable polygon. As shown in the example of FIG. 1, a cylinder 112 may have substantially straight and parallel sides. However, in some embodiments of the present disclosure, the sides of the cylinder 112 may be tapered and/or the width 117 may vary as a function of position along the y-axis. Among other things, varying a width 117 and/or cross-sectional profile of a reactor 110 may be utilized to promote mixing of the contents of the reactor 110 (e.g. algae suspended in a growth medium).

In some embodiments of the present disclosure, the height 116 of the cylinder 112 of a reactor and the width 117 of a cylinder 112 may have a height to width ratio (H:W) between about 2:1 and about 6:1, or between about 3:1 and about 5:1. In some embodiment of the present disclosure, the ratio of height to width may be about 4:1. In some embodiments of the present disclosure, the height 116 of the cylinder 112 of a reactor 110 may be between about 10 cm and about 160 cm, or between about 20 cm and about 40 cm. In some embodiments of the present disclosure, the width 117 of a cylinder 112 of a reactor 110 may be between about 5 cm and about 50 cm. In some embodiments of the present disclosure, a reactor 110 may scale-up to a cylinder 112 having a height 116 up to two meters, with a width 117 up to 60 cm.

In some embodiments of the present disclosure, the bottom portion 114 may be substantially rounded. For example, the bottom portion 114 of a reactor 110 may be in the shape of a spherical head, a hemispherical head, a semi-elliptical head, a torispherical head, a flat dished head, a dished disc, and/or a toriconical head. Any rounded and/or tapered bottom portion 114 may be used that provides a space for the mixer 140 to enable mixing of the reactor contents (not shown); e.g. mixing that prevents settling of solids into the bottom portion 114 of the reactor 110.

The top portion 113 of a reactor 110 may also be rounded, as described above regarding the bottom portion 114 of the reactor 110. In some embodiments of the present disclosure, a top portion 113 may not be rounded and may be constructed of a relatively flat, planar piece of material. A neck 115 may be provided as an integral part of the top portion 113 of a reactor 110, where the neck 115 may be configured with a hole to enable access to the internal volume of the reactor 110, wherein the “internal volume” includes the space defined by the wall of the cylinder 112, the bottom portion 114, and the top portion 113. Like the cylinder 112, a neck 115 may be cylindrical in shape, with a substantially circular and/or elliptical cross-section in the xz-plane. However, a neck 115 may define any other cross-sectional shape, as suited for a particular application. A neck 115 may be configured to receive a cap 120, where the cap 120 provides a liquid-tight and/or gas-tight seal with the neck 115 of the reactor 110. A tight seal where the cap 120 interfaces with the neck 115 may be needed to prevent the contents of the reactor 110 from being polluted by external contaminants (e.g. bacteria, viruses, yeast, etc.). Thus, in some embodiments of the present disclosure, a cap 120 may be a rubber stopper or a screw-on cap. As shown in FIG. 1, a cap 120 may include at least one port 125 configured to receive a tube 130 such that the tube 130 may be inserted to a desired position within the internal volume of the reactor 110. The distal end of a tube 130 may be positioned within the internal volume formed by the rounded bottom portion 114, within the internal volume formed by the cylinder 112, and/or positioned within the internal volume formed by the top portion 113. In some embodiments of the present disclosure, a reactor unit 100 may include a cap 120 having more than one port 125, e.g. two ports, three ports, four ports, or more, as needed for a particular application. A port 125 provides a route for supplying materials to the reactor 110, such as nutrients, as well as a route for removing waste material from the reactor, such as gas produced by an organism's (e.g. algae) metabolism. In some embodiments of the present disclosure, when scaling up to larger sizes, the top portion 113 may include built-in glass ports 125 for feeding gas into and out of the reactor, as well as a built in glass sample port and a connection for an internal light source (not shown; more on this below).

Referring again to FIG. 1, parts of a reactor 110 or the entirety of a reactor 110 may be constructed of a material that is substantially transparent to light in the visible spectrum and/or transparent to light having a wavelength between about 400 nm and 700 nm. This is advantageous for examples where a light source is external to the reactor 110. In some embodiments of the present disclosure, a reactor 110 may be constructed of at least one of glass, plexiglass, quartz, and/or any suitably transparent plastic. In some embodiments of the present disclosure, a reactor may be constructed using a suitable metal, e.g. stainless steel, that is not transparent to light. Use of a metal to construct a reactor may be advantageous when scaling up to larger reactor sizes, which may utilize a light source positioned within the reactor itself. In some embodiments of the present disclosure, the entire reactor unit 100 may be autoclavable (e.g. capable of withstanding temperature to at least 121° C., or in a range between about 100° C. and about 130° C.). In some embodiments of the present disclosure, a port 125 in a cap 120 may be modified to accommodate something other than a tube; e.g. transmitter and/or receiver cables for instrumentation, electrical cords (e.g. for an internal light source), etc. In some embodiments of the present disclosure, a cap 120 may have a portion of its outer circumference modified to allow the passage of a tube, cord, and/or cable between the outer surface of the cap and the inner surface of the neck 115, while still maintaining a gas-tight and/or liquid-tight seal.

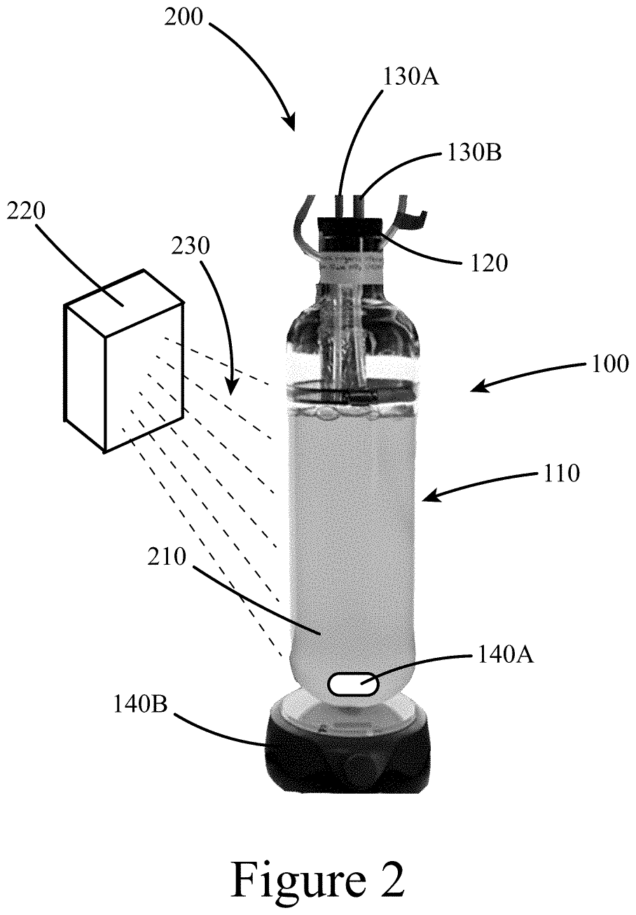

FIG. 2 illustrates a reactor system 200 utilizing a reactor unit 100 like that shown in FIG. 1 and described above. In this example, the reactor 110 of the reactor unit 100 contains a reaction medium 210, which is illuminated with light 230 by a light source. In some embodiments, especially when scaling up to larger sized reactors, a light source 220 may be positioned within the reactor 110 (internal light source not shown). Further, in this example, the mixer includes two parts 140A and 140B, where 140A is a cylindrically shaped magnetic stir bar magnetically coupled to an adjustable magnetic drive 140B. In this example, the reaction medium 210 includes an algae strain suspended in a growth medium. The reactor unit 100 includes at least two tubes, a gas supply tube 130A and gas return tube 130B.

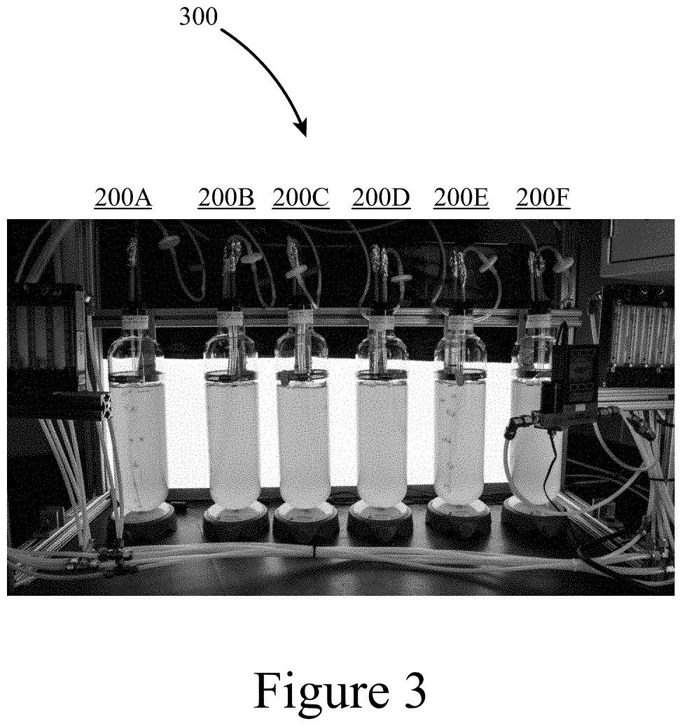

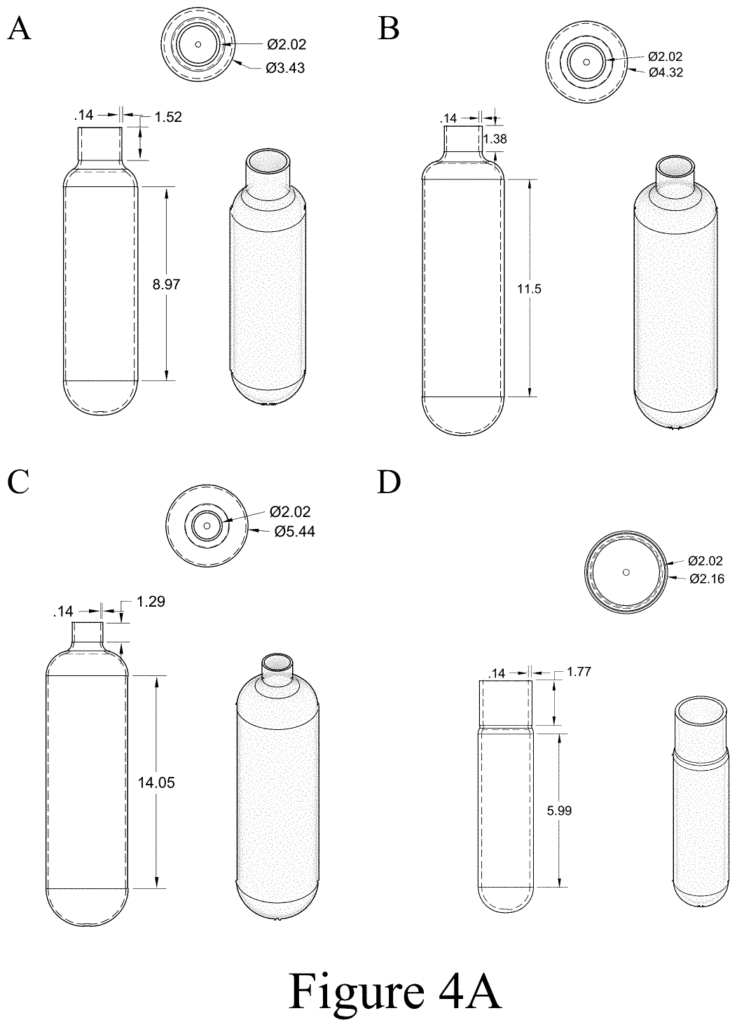

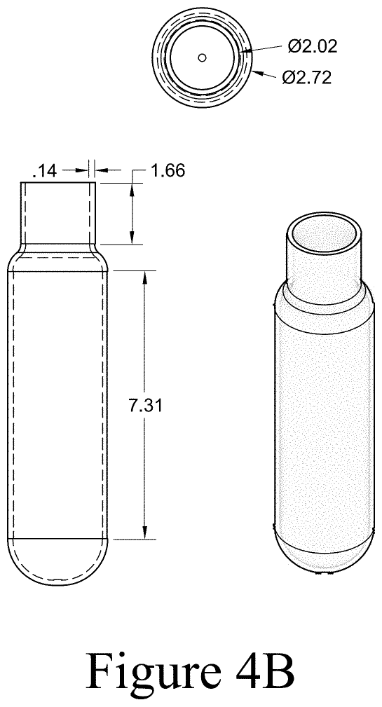

FIG. 3 illustrates a reactor array 300 that includes six individual reactor systems (200A-200F), each system including its own dedicated mixer and supply/return systems. FIGS. 4A and 4B illustrate examples of reactors, according to some embodiments of the present disclosure, having various configurations, geometries, and dimensions. These illustrate that, in some embodiments of the present disclosure, a reactor may have a cylinder having a straight-side height between 6 inches and 14 inches and a width between 2.16 inches and 5.44 inches. In some embodiments of the present disclosure, a reactor may have a neck having a height between 1.3 inches and 1.75 inches and a width of about 2 inches. However, these examples are more illustrative for reactors and reactor systems designed for the laboratory scale. As described above, in some embodiments of the present disclosure, reactors and reactor systems may be designed to have significantly larger dimensions.

### Example

Synechocystis sp. PCC 6803 wild-type (WT) and flv1 and flv3 knockout strains were maintained on BG-11 (Allen, 1968) supplemented with 20 mM NaHCO₃ and 20 mM TES (pH 6.8). Growth medium was supplemented with 25 mg L−1 spectinomycin for flv1KO, and 100 mg L−1 kanamycin for flv3KO. Maintenance cultures were grown in a Percival chamber in 250 mL flasks at 30 C, 50 μE white LED light, in air supplemented with 5% CO₂ and with shaking (120 rpm). For experimental conditions, varied light conditions were used: 35 μE continuous light (GL 35), and fluctuating light (35 μE, 5 min/500 μE, 30 s) (FL 35/500). To assess growth in FL 35/500, cultures were first grown in GL 35 for 3 days (approximately 0.7 to 1.2 OD 730 nm), before switching to FL 35/500. Under these conditions, cultures were grown in a reactor, as described herein, having a cylinder portion with a height of 40 cm and a width of 13 cm and with a round bottom portion and a round top portion with a narrow neck. The bottles were fitted with a cap (i.e. rubber stopper) having three ports to accommodate a first tube for gas input, a second tube for gas output, and a third tube for sample collection during growth. Cultures were grown at room temperature, in air supplemented with 3% CO₂, agitated using stirring (0.5 inch stir bar) and bubbling, and were illuminated from the side using white LED panels.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

What is claimed is:

1. A reactor comprising:
a cylinder integrally connected to a top portion and a bottom portion, wherein:
the bottom portion is rounded and configured to contain a stir bar,
the top portion comprises a neck configured to receive a cap,
the cylinder has a height (H) and a width (W) such that the ratio of H to W is between about 1:1 and about 10:1, and
the cylinder is transparent to light having a wavelength between about 400 nm and about 700 nm.

2. The reactor of claim 1, wherein the ratio is between about 2:1 and about 6:1.

3. The reactor of claim 1, wherein H is between about 5.5 inches and about 14.5 inches.

4. The reactor of claim 1, wherein W is between about 2.0 inches and about 5.5 inches.

5. The reactor of claim 1, wherein the neck has a height between 1.0 inch and 2.0 inches.

6. The reactor of claim 1, wherein the neck has a width of about 2 inches.

7. The reactor of claim 1, wherein the cylinder is constructed of at least one of glass, plexiglass, quartz, or a plastic.

8. The reactor of claim 1, wherein the reactor is capable of withstanding a temperature between about 100° C. and about 130° C.

9. The reactor of claim 1, wherein the cylinder has a wall thickness of about 0.14 inches.

10. A reactor system comprising:
a reactor comprising:
a cylinder integrally connected to a top portion and a bottom portion, wherein:
the bottom portion is rounded,
the top portion comprises a neck,
the cylinder has a height (H) and a width (W) such that the ratio of H to W is between about 1:1 and 10:1, and
the cylinder is transparent to light having a wavelength between about 400 nm and about 700 nm;
a stir bar positioned in the bottom portion;
a cap positioned within the neck; and
a first tube positioned in a first port passing through the cap into the reactor.

11. The reactor system of claim 10, further comprising a light source configured to provide light having a wavelength between about 400 nm and about 700 nm.

12. The reactor system of claim 10, wherein a distal end of the first tube is positioned in the bottom portion.

13. The reactor system of claim 10, further comprising a second tube positioned in a second port passing through the cap into the reactor.

14. The reactor system of claim 13, wherein a distal end of the second tube is positioned in the bottom portion.

15. The reactor system of claim 12, further comprising a third tube positioned in a third port passing through the cap into the reactor.

16. The reactor system of claim 15, wherein a distal end of the third tube is positioned in the top portion.

17. The reactor system of claim 10, wherein the stir bar is magnetic.

18. The reactor system of claim 17, further comprising a stirring mechanism that is magnetically coupled to the stir bar and drives rotation of the stir bar.

19. A method comprising:
positioning a mixture comprising an algae and a growth medium in a reactor;
agitating the mixture using a mixer positioned in the reactor at a speed between greater than zero RPM and about 1000 RPM;
providing a gas to the mixture; and
illuminated the mixture using a light source positioned externally to the reactor.

20. The method of claim 1, wherein the light source is configured to provide at least one of a steady light or a fluctuating light.
